(19) United States
(12) Patent Application Publication
(10) Pub. No.: US 2026/0210948 A1
(43) Pub. Date: Jul. 23, 2026

(54) FLUORESCENCE-BASED ASSAY FOR IDENTIFICATION OF PROTAC LIGANDS FOR HECT-TYPE E3 LIGASES

(71) Applicant: Purdue Research Foundation, West Lafayette, IN (US)

(72) Inventors: Sebastian Kenny, West Lafayette, IN (US); Chittaranjan Das, West Lafayette, IN (US)

(73) Assignee: Purdue Research Foundation, West Lafayette, IN (US)

(21) Appl. No.: 19/139,486

(22) PCT Filed: Dec. 15, 2023
(86) PCT No.: PCT/US2023/084294

§ 371 Date: Jun. 16, 2025

Related U.S. Application Data
(60) Provisional application No. 63/433,172, filed on Dec. 16, 2022.

(51) Int. Cl. G01N 33/542; C12Q 1/25; G01N 21/64; G01N 33/68

(52) U.S. Cl. CPC G01N 33/542; C12Q 1/25; G01N 21/6445; G01N 33/6845; G01N 2333/9015; G01N 2440/36

(57) ABSTRACT

A method of identifying a ligand, which is a ligand for a target protein of interest, and which interacts with a homologous to E6AP C-terminus (HECT)-type E3 ligase; a composition comprising (a) a fluorescently labeled peptide comprising a PPxY motif, an E1 ubiquitin (Ub)-activating enzyme, an E2 Ub-conjugating enzyme, a HECT-type E3 ligase, Ub, adenosine triphosphate (ATP), and a reaction buffer or (b) a fluorescently labeled peptide comprising a PPxY motif, a Ub-charged E2 Ub-conjugating enzyme, a HECT-type E3 ligase, ATP, and a reaction buffer; and a composition comprising a ligand identified in accordance with the above method or a pharmaceutically acceptable salt thereof.

### CROSS-REFERENCE TO RELATED APPLICATIONS

This application claims priority to U.S. provisional patent application No. 63/433,172, filed Dec. 16, 2022.

### TECHNICAL FIELD

The present disclosure relates to ubiquitination, fluorescence polarization, PROTAC ligands, and Hect-type E3 ligases.

### BACKGROUND

Protein ubiquitination involves attachment of ubiquitin (Ub) to proteins. It is a conserved, essential, and highly organized mechanism of post-translational regulation of diverse cellular processes, ranging from proteasomal degradation to protein trafficking, cell-cycle regulation, and immune signaling (Komander et al., Annu Rev Biochem 81: 203-229 (2012)).

Ub is typically attached to a lysine residue of a target protein—a mono-ubiquitin (monoUb) modification—and more than one lysine residue in a target protein can be ubiquitinated. Ubiquitination is usually not a singular modification, but generally involves the subsequent attachment of the C-terminus of one Ub molecule to a lysine of another Ub moiety (positions K6, K11, K27, K29, K33, K48 and K63), giving rise to polyubiquitin chains (polyUb) with distinct Ub linkages, each playing distinct roles in cellular homeostasis. For example, polyUb linkages mediated by Ub residue K48 (K48-linked chains) usually designate the protein for proteasomal degradation, whereas polyUb linkages mediated by residue K63 (K63-linked chains) mediate endo-lysosomal trafficking associated with cell-surface receptor signaling and membrane protein turnover. Such a tightly regulated protein quality control mechanism is essential for maintaining cellular protein homeostasis or proteostasis.

In humans, ubiquitination occurs via a cascade of Ub “writing” enzymes, which include an E1 Ub-activating enzyme, an E2 Ub-conjugating enzyme, and an E3 Ub ligase. The E1 Ub-activating enzyme, expending adenosine triphosphate (ATP), creates a high-energy thioester linkage between a cysteine residue in the E1 active site and the carboxy terminus of Ub. Ub is then transferred from the E1 Ub-activating enzyme to a cysteine residue in the E2 Ub-conjugating enzyme in a transthiolation reaction. The Ub can is then transferred onto a lysine residue of a target protein in a reaction catalyzed by an E3 Ub ligase either directly, in the case of the RING/U-box family of E3 Ub ligases, or indirectly, in the case of HECT or RBR families, in which the Ub is transferred onto a lysine residue of a target protein via thioester intermediate at a cysteine residue in the catalytic site of the ligase.

The resulting ubiquitination signals are “read” by Ub binding domains, which direct cellular processes in view of what is “read.” The ubiquitination signals can be “erased” by deubiquitinases, which can edit or recycle the Ub signal back to monoUb.

Approximately 5% of human genes encode regulators of Ub signaling. Faulty regulation can often lead to disease (Popovic et al., Nat Med 20: 1242-1253 (2014)). Defects in Ub signaling are linked to many cancers. For example, dysregulation of the E3 Ub ligase can affect the stability of tumor suppressors or oncogene products (Kirkin et al., Curr Opin Genet Dev 21: 21-28 (2011)). Defects in the Ub proteasome system can lead to an inability to degrade toxic protein aggregates, which in turn can result in neurodegeneration (Zheng et al., Front Aging Neurosci 8: 303 (2016)). Aberrant ubiquitination can result in constitutive activation of signaling pathways, which in turn can result in autoimmune disease or tumors (Hu et al., Cell Res 26: 457-483 (2016)).

In view of the above, pharmaceutical research has recently focused on the development of small molecule effectors of targeted protein degradation (TPD), which coopt the mechanisms of E3 Ub ligases to target undruggable proteins for induced ubiquitination and proteasomal degradation. Two classes of TPD effectors developed to make such use of induced ubiquitination include PROteolysis TArgeting Chimeras (PROTACs) and molecular glues. Both type ubiquitination-induced effectors employ engineered organic molecules designed to interact with a targeted protein and an E3 Ub ligase. PROTACs are bivalent molecules that consist of an E3 ligase recruiter linked to a protein-targeting ligand and function to induce the formation of ternary complexes that bring together an E3 ubiquitin ligase and the target protein as a novel substrate for ubiquitination and subsequent degradation. Because PROTACs do not rely on catalytic site targeting, PROTACs have the potential to target a wide array of previously undruggable proteins. However, the design of PROTACs so far has relied on a limited number of well-characterized E3 ligases (viz., Cullin RING family), most of which are cytosolic or nuclear, and rely on complex formation with ligase-specific adaptors for activation.

High-throughput screening (HTS) of ligands for potential protein targets is critical for the development of PROTACs and other small effectors of targeted protein degradation (TPD) and other ubiquitination activities. However, despite the recent focus on developing TPD effectors, there are presently no viable assays or screening methods involving membrane-bound E3 Ub ligases, which has so far prevented the development of ubiquitination modulators for targeting of oncogenic receptors and other proteins linked to dysregulation of membrane homeostasis, many of which are considered undruggable.

### SUMMARY

A method of identifying a ligand, which is a ligand for a target protein of interest, and which interacts with a homologous to E6AP C-terminus (HECT)-type E3 ligase, is provided. The method comprises:
(a) incubating the ligand with:
   (i) a fluorescently labeled peptide comprising a short PY motif and
   (ii) (a′) an E1 ubiquitin (Ub)-activating enzyme, an E2 Ub-conjugating enzyme, the HECT-type E3 ligase, Ub, and adenosine triphosphate (ATP) or
      (b′) a Ub-charged E2 Ub-conjugating enzyme, the HECT-type E3 ligase, and ATP and
(b) measuring fluorescence polarization (FP) over time, whereupon:
if the ligand interacts with the HECT-type E3 ligase, the fluorescently labeled short PY peptide is displaced from binding to the HECT-type E3 ligase, and there is no change in FP over time and
if the ligand does not interact with the HECT-type E3 ligase, the fluorescently labeled peptide comprising a PY motif is ubiquitinated, and there is a change in FP over time,
whereupon a ligand that interacts with a HECT-type E3 ligase is identified.

The HECT-type E3 ligase according to examples can be a membrane-bound type E3 ligase of the human NEDD4 subfamily of HECT-type E3 ligases. For instance, the HECT-type E3 ligase can be one of NEDD4-1, NEDD4-2 (NEDD4L), ITCH, WWP1, WWP2, SMURF1, SMURF2, HECW1 (NEDL1), or HECW2 (NEDL2), including any isomers thereof.

According to certain examples, the HECT-type E3 ligase can be a NEDD4 hybrid human-ortholog construct (NEDD4_{HYBRID}). For instance, a NEDD4_{HYBRID} consistent with the disclosure may include an orthologue HECT domain and one or more NEDD4 WW domains; one or more ortholog linkers and one or more NEDD4 WW domains; ortholog C2 domains and one or more NEDD4 domains; or any combination of ortholog HECT domain, linkers, and C2 domain with one or more NEDD4 WW domains. In certain examples, the NEDD4 ortholog is expressed as a fungal HECT-type E3 ligase. For example, the NEDD4 homologue can be yeast (S. cerevisiae) Rsp5, or yeast (S. Pombe) Pub1, Pub2, or Pub3.

In one such example, a NEDD4_{LHYBRID} can comprise an Rsp5 surrogate in which the native WW1, WW2, and WW3 domains are substituted, respectively, with human NEDD4L WW2, WW3, and WW4 domains. In the example, the Rsp5 surrogate can include a native C2 domain or the native C2 domain may be omitted.

Short PY motifs consistent with the disclosure can include PPxY and LpxY motifs, where x can be any amino acid. In examples, the fluorescently labeled peptide comprising a PY motif can be a fluorescently labeled peptide having an amino acid sequence from yeast vesicular snare protein (Sna4) and a PPxY and/or LPxY motif insert.

A composition is also provided. The composition comprises (a) a fluorescently labeled peptide comprising a PPxY motif, an E1 Ub-activating enzyme, an E2 Ub-conjugating enzyme, a HECT-type E3 ligase, Ub, adenosine triphosphate (ATP), and a reaction buffer or (b) a fluorescently labeled peptide comprising a PPxY motif, a Ub-charged E2 Ub-conjugating enzyme, a HECT-type E3 ligase, ATP, and a reaction buffer.

Another composition is further provided. The composition comprises the ligand identified in accordance with the above method or a pharmaceutically acceptable salt thereof.

### BRIEF DESCRIPTION OF THE FIGURES

FIG. 1 is a schematic drawing of the domains of the human E3 ligase NEDD4L.

FIG. 2 is a schematic drawing of an assay design to identify tight binders.

FIG. 3A shows the results of the ubiquitination assay as monitored by fluorescence polarization.

FIG. 3B shows that the ubiquitination can be reversed by the addition of de-ubiquitinase.

FIG. 3C shows the specificity of the binding between the PPxY motif and the WW domain as evidenced by PPxY mutant analysis.

FIG. 3D shows the sensitivity of the binding to increasing levels of the inhibitor iodoacetamide.

FIG. 4A shows that NEDD4L-Rsp5HYBRID has an increased initial rate compared to Rsp5.

FIG. 4B shows that NEDD4L-Rsp5HYBRID detects the Sna4 peptide with similar selectivity as Rsp5 (WT).

### DETAILED DESCRIPTION

The present disclosure is predicated, at least in part, on the development of an assay for identification of PROTAC ligands utilizing “homologous to E6AP C-terminus” (HECT)-type E3 ligases, also referred to as HECT-type E3 ubiquitin transferases (see, e.g., IntEnz EC 2.3.2.26). HECT-type E3 ligases play a central role in the regulation of proteostasis in various conditions including cancers and neurodegenerative disorders.

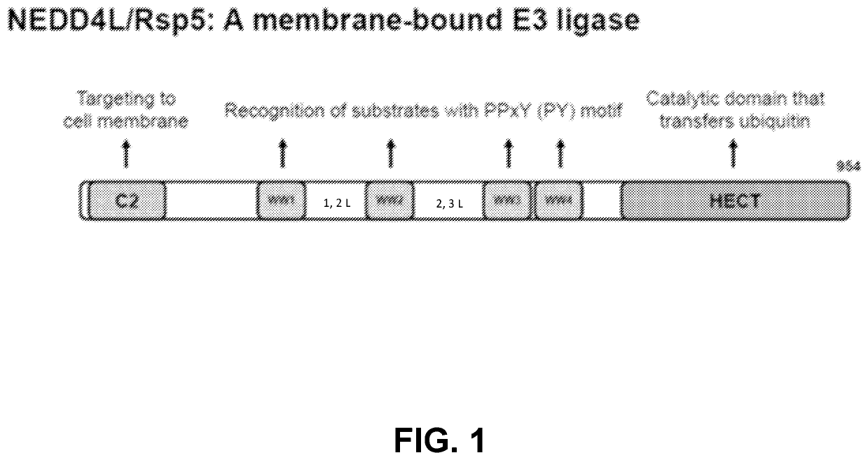

The human HECT-type E3 family consists of 28 members, which can be divided into three subfamilies based on their N-terminal domain architecture: the NEDD4 subfamily (9 members), the HERC subfamily (6 members), and other HECTS (13 members). The nine members of the human NEDD4 subfamily are NEDD4-1, NEDD4-2 (NEDD4L), ITCH, WWP1, WWP2, SMURF1, SMURF2, HECW1 (NEDL1), and HECW2 (NEDL2). With reference to FIG. 1 (NEDD4L), the human NEDD4 subfamily are membrane-bound E3 ligases characterized by (a) an N-terminal C2 domain, which binds to phospholipids and mediates intracellular targeting to the plasma membrane, endosomes and multivesicular bodies; (b) two-to-four WW domains, which bind to PY motifs in substrate proteins; (c) domain linkers (e.g., 1, 2 and 2, 3 WW domain linkers of NEDD4L, as illustrated in FIG. 1); and (d) a C-terminal HECT domain, which serves as the catalytic site for protein ubiquitination.

The WW domain is a protein-protein interaction module containing W-21x-W motif of two conserved tryptophan residues spaced apart by 21 residue linker that folds into a meandering triple-stranded beta sheet connected laterally by at least two or three backbone hydrogen bonds. The WW domain mediates interactions with ligand substrates with short proline-containing motifs (PPxY, LPxY), or phosphor-threonine or phosphor-serine residues, forming regulatory ubiquitin complexes in various signaling networks. The human NEDD4 subfamily has four domain classes (WW1-WW4). Individual E3 ligases may include tandem WW domains for increased selectivity for dual-motif ligands or multiple independent WW domains with distinct binding preferences supporting broad functional diversity.

The C-terminal HECT domain consists of approximately 350 amino acid residues conformed in a bilobed structure with a larger N-terminal lobe (N-lobe) that contains the E2 binding site, and a smaller C-terminal lobe (C-lobe) carrying a catalytic cysteine. The HECT domain directly catalyzes the covalent bond between Ub and a targeted substrate through a two-step reaction. In the first step, the HECT domain captures Ub from the E2 ubiquitin-conjugating enzyme (IntEnz EC 2.3.2.23) to the catalytic cysteine located in the C-terminal lobe through transthiolation. In the second step, the HECT domain transfers the ubiquitin to the target substrate, resulting in the formation of an isopeptide bond between the C-terminal glycine residue of ubiquitin and the s-amino group of an L-lysine residue of the target.

Despite their common features, the various NEDD4 subfamily members of HECT-type E3 ligases exhibit unique functions. For example, NEDD4-1 is a positive regulator of cell surface insulin-like growth factor (IGFIR) signaling, negative regulator of select cytokine and chemokine responses elicited by TRIF-independent Toll-like receptors (TLRs), and it controls the sorting of transmembrane proteins (e.g., LAPTM5) and vesicular sorting of cargo proteins. NEDD4L promotes ubiquitination of various plasma membrane channels (e.g., the epithelial Na+ channel (ENaC)), their endocytosis from the plasma membrane and degradation, downregulates autophagy and cell growth by ubiquitinating and reducing cellular ULK1 or ASCT2 levels, and promotes ubiquitination and degradation of SGK1 and TNK2 serine/threonine protein kinases implicated in cell spreading and migration, cell survival, cell growth and proliferation.

SMURF1 and SMURF2 antagonize TGFβ and BMP signaling by ubiquitinating TGFBR1 and targeting it for degradation. ITCH controls the levels of JUNB and JUN substrates, which regulate the transcription of the interleukin 4 (IL4) gene, controls the level of mediated tumor necrosis factor-α (TNFα)-induced apoptosis through degradation of FLIP, and controls the levels of notch substrate, which cause cell cycle arrest and apoptosis in response to DNA damage. WWP1 ubiquitinates ERBB4 isoforms JM-A CYT-1 and JM-B CYT-1, KLF2, KLF5 and TP63 and promotes their proteasomal degradation, and ubiquitinates and promotes degradation of TGFBR1. WWP2 polyubiquitinates POU5F1 and promotes it to proteasomal degradation, regulates activation-induced cell death in T cells through ubiquitination and promoting degradation of EGR2, ubiquitinates SLC11A2; and Ubiquitinates RPB1 and promotes it to proteasomal degradation. HECW1 ubiquitinates and promotes degradation of DVL1 and targets the mutant SOD1 protein involved in familial amyotrophic lateral sclerosis (FALS). HECW2 ubiquitinates TP73 to enhance activation of transcription by TP73.

Because the NEDD4 subfamily of E3 ligases localizes onto the plasma membrane through the C2 domain, they may be utilized to target numerous membrane proteins outside of their canonical substrates, including, for example, (1) receptor tyrosine kinases, like VEGF-R and TrkB, implicated in cancers, diabetes, inflammation, severe bone disorders, arteriosclerosis and angiogenesis, lung fibrosis, (2) nuclear receptors, like RXR, implicated in Alzheimer's disease, Parkinson's disease, Huntington's disease, ALS, and (3) cytochrome p450 proteins, like CYP1 and CYP51, implicated in genital anomalies, disordered steroidogenesis, hormonal changes, cholesterol. In that regard, members of the NEDD4 subfamily of E3 ligases may be harnessed to downregulate membrane proteins that are aberrant/upregulated in diseases via the ubiquitination and degradation signal path.

In one aspect, methods of identifying a ligand for a target protein of interest are provided. For instance, a method is provided that includes identifying a ligand for a target protein of interest based on the detection of an interaction between a subject ligand and a HECT-type E3 ligase. In one example, the method may comprise:
(a) incubating a subject ligand with:
   (i) a fluorescently labeled peptide comprising a PY motif and
   (ii) either (a′) an E1 ubiquitin (Ub)-activating enzyme, an E2 Ub-conjugating enzyme, the HECT-type E3 ligase, Ub, and adenosine triphosphate (ATP) or
      (b′) a Ub-charged E2 Ub-conjugating enzyme, the HECT-type E3 ligase, and ATP and
(b) measuring fluorescence polarization (FP) over time, whereupon:
if the ligand interacts with the HECT-type E3 ligase, the fluorescently labeled PY peptide is displaced from binding to the HECT-type E3 ligase, and there is no change in FP over time and
if the ligand does not interact with the HECT-type E3 ligase, the fluorescently labeled peptide comprising a PY motif is ubiquitinated, and there is a change in FP over time,
whereupon a ligand that interacts with a HECT-type E3 ligase is identified.

In certain examples, the method of identifying a ligand for a target protein of interest includes identifying a ligand for a target protein of interest based on the detection of an interaction between a subject ligand and a ligase member of the human NEDD4 subfamily of HECT-type E3 ligases, including any isoforms thereof. Example natural HECT-type E3 ligases of the human NEDD4 subfamily include NEDD4-1 (alias KIAA0093; HGNC:7727); NEDD4-2 (NEDD4L) (alias KIAA0439; HGNC:7728); ITCH (HGNC:13890); WWP1 (HGNC:17004); WWP2 (HGNC:16804); hSMURF1 (alias KIAA1625; HGNC:16807); hSMURF2 (HGNC:16809); HECW1 (alias KIAA0322; HGNC:22195); HECW2 (alias KIAA1301; HGNC:29853).

According to certain examples, the HECT-type E3 ligase can be a NEDD4 hybrid human-ortholog construct (NEDD4HYBRID). For instance, a NEDD4HYBRID consistent with the disclosure may include an orthologue HECT domain and one or more NEDD4 WW domains; one or more ortholog linkers and one or more NEDD4 WW domains; ortholog C2 domains and one or more NEDD4 domains; or any combination of ortholog HECT domain, linkers, and C2 domain with one or more NEDD4 WW domains. In certain examples, the NEDD4 ortholog can be obtained from a microorganism of the subphylum Saccharomycotina. For example, the NEDD4 homologue can be yeast (S. cerevisiae) Rsp5, or yeast (S. Pombe) PubI, Pub2, or Pub3. Rsp5 may be particularly apt as a surrogate for certain applications contemplated herein because Rsp5 (1) is stably expressed in conventional bacterial expression systems; (2) includes HECT and C2 domains that are structurally and functionally similar to human NEDD4 ligases; and (3) has similar recognition of the PPxY motif.

In one such example, a method is provided for identifying a ligand for a target protein of interest that interacts with NEDD4L ligase based on the detection of an interaction between a subject ligand and an analogous NEDD4LHYBRID:
(a) incubating a subject ligand with:
   (i) a fluorescently labeled peptide comprising a PY motif and
   (ii) either (a′) an E1 ubiquitin (Ub)-activating enzyme, an E2 Ub-conjugating enzyme, a NEDD4LHYBRID, Ub, and adenosine triphosphate (ATP) or
      (b′) a Ub-charged E2 Ub-conjugating enzyme, the NEDD4LHYBRID, and ATP, and
(b) measuring fluorescence polarization (FP) over time, whereupon:
if the ligand interacts with the NEDD4LHYBRID, the fluorescently labeled PY peptide is displaced from binding to the NEDD4LHYBRID, and there is no change in FP over time and
if the ligand does not interact with the NEDD4LHYBRID, the fluorescently labeled peptide comprising a PY motif is ubiquitinated, and there is a change in FP over time,
whereupon a ligand that interacts with the NEDD4LHYBRID is identified as a putative ligand that interacts with a NEDD4 E3 ligase.

In one such example, a NEDD4LHYBRID may comprise an Rsp5 surrogate with native HECT domain and domain linkers (Rsp5HECT) in which the native WW1, WW2, and WW3 domains are substituted, respectively, with human NEDD4L WW2, WW3, and WW4 domains (NEDD4L_{WW2, WW3, WW4}-Rsp5_{HECT} or NEDD4L-Rsp5_{HYBRID}). NEDD4L may be particularly apt for combination with Rsp5 for certain applications herein due, at least in part, to the number of native substrates shared in common, including H+/Cl− exchange transporter, Na+channel protein, Cdc42-associated tyrosine kinase, PIK3CA protein, and receptor tyrosine kinases. In any of the foregoing examples, the Rsp5 surrogate may include a native C2 domain or the native C2 domain may be omitted.

The labeled peptide comprising a PY motif, such as a fluorescently labeled peptide comprising a PPxY or LPxY motif, can be a labeled, such as fluorescently labeled, peptide having an amino acid sequence from yeast vesicular snare protein (Sna4) and containing a PPxY motif. The peptide comprising a PPxY motif can be labeled by any suitable method known in the art, depending on the detection method to be used (e.g., fluorescence, chemiluminescence, enzymatic, or radioactivity). In examples, the labeled peptide has only a single PPxY motif and the peptide binds to a single WW domain. In other examples, the labeled peptide has a dual PPxY motif and the peptide binds to tandem WW domains.

Exemplary fluorescent moieties include, but are not limited to, fluorescein, lissamine, phycoerythrin, rhodamine, Cy2, Cy3, Cy3.5, Cy5, Cy5.5, Cy7, FluorX, Texas Red, Alexa Fluor, Oregon Green, indocyanine green, malachite green, BODIPY, dansyl, tetramethyl rhodamine, benzoxadioazole, coumarin, eosin, Lucifer Yellow, pyridyloxazole, and derivatives thereof. See, e.g., Handbook of Fluorescent Probes and Research Chemicals (Molecular Probes, Inc. (2000)). Such labels can be attached to the peptide comprising a PPxY motif, for example, by any suitable means, such as covalent or noncovalent linkages or chemical cross-linking.

Detection methods include, but are not limited to, scintillation proximity assay (SPA), chemiluminescence, fluorescence intensity (FLINT), FRET, and TR-FRET. The terms “detect” and “detection” are used to refer to a determination of interaction between the ligand to a protein of interest and the E3 ligase. The detection can be by any suitable means as known in the art. Thus, the label can be fluorescent, radioactive, enzymatic, or chemiluminescent.

In example assays, when a single WW domain of the E3 ligase binds a PY motif in the ligand, the HECT domain ubiquitinates the ligand. In that regard, PY binding can be used to measure ligand efficacy. In one example assay, a fluorescence polarization (FP)-based assay may be used in which a peptide substrate conjugated with a fluorophore is bound, via a PY binding site, to a WW domain of an E3 ligase. Baseline fluorescence under excitation is measured. Peptide-bound E3 ligate are contacted with a compound to be screened for binding to the occupied WW domain. If the compound can bind to a WW domain of the E3 ligase, it displaces the fluorescently labeled peptide substrate, and there is no change in fluorescence. If the compound cannot bind to a WW domain of the E3 ligase, it does not displace the fluorescently labeled Peptide substrate, the peptide substrate is ubiquitinated, and fluorescence increases over time. The FP-based assay may be further utilized to quantitate substrate recruitment.

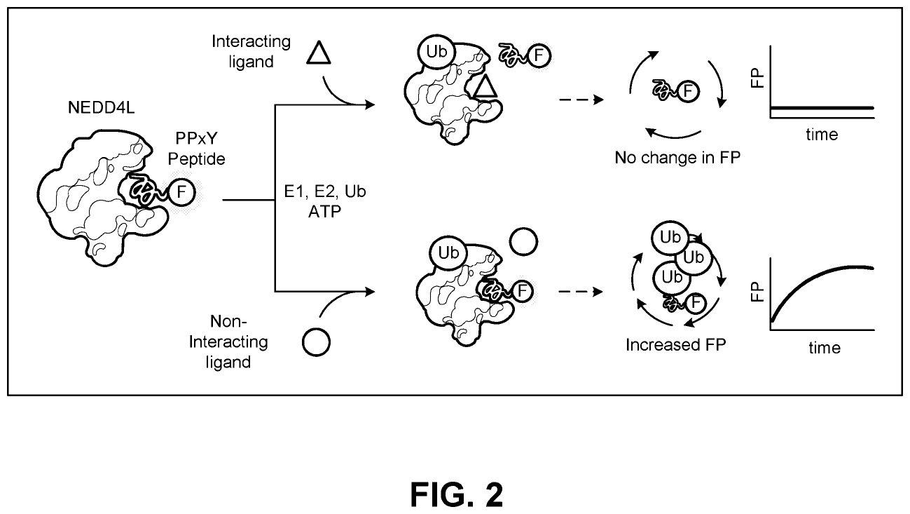

In examples, a high-throughput assay platform is provided for screening for ligands that can be optimized for binding to the WW domains of a NEDD4 subfamily member or NEDD4 hybrid which target membrane receptors bearing PY motifs, including PPxY peptides. By observing the displacement of a previously characterized tight-binding PPxY peptide via fluorescence polarization (KD=4.5 mM), fragments that have the potential to be developed for ligase ligands for these WW-domain containing HECT-type E3 ligases can be identified (see FIG. 2). Ubiquitination activity by the HECT domain catalytic cysteine can be measured by direct comparison of peptide ubiquitination. Fragments that are tight binders tend to displace the labeled peptide from the WW-domain, which in turn would inhibit ubiquitination of the peptide, leading to a lower FP (Fluorescence Polarization) compared to a non-binder to the WW-domain. In sum, if the peptide interaction with the WW-domain is interfered with by a chemical fragment, the peptide will not be recruited to the E3 ligase, hence no ubiquitination will be observed. Chemical fragments that cause no ubiquitination or a lower rate of ubiquitination can be used as candidate molecules for ligase-binding ligand development.

In certain embodiments, the formation of ubiquitin complexes may be measured by an interactive method, such as fluorescence resonance energy transfer (FRET). By way of example, Ub can be labeled with a first fluorescent label and the ligand can be labeled with a second fluorescent label. The first and second labels interact when they come into close proximity to produce an altered signal. Polyubiquitination can be detected by mixing two or more pools of differentially labeled Ub. High throughput screening of ligands for a target protein of interest that can interact with a HECT-type E3 ligase can be achieved in solution. The tagged ligand can be isolated. Ligands also can be resolved by SDS-PAGE for detection.

In certain embodiments, time-resolved FRET (TR-FRET) can be used. TR-FRET is based on the proximity of a donor label and an acceptor label, which have been brought together by a specific binding reaction. The excited energy of the donor-chelate is transferred by a nonradiative resonance energy transfer mechanism to an acceptor within a short distance. Fluorescent lanthanide chelates (e.g., europium chelate, terbium chelate, fluorescein, europium cryptate) are typically used to avoid interference caused by short-lived emission from acceptor molecules excited directly, rather than by energy transfer.

Currently, the majority of ligases utilized for PROTACs fall under the family of Cullin RING ligases (CRLs), which are responsible for turnover of nuclear and cytosolic proteins in site-specific manner, e.g., chromatin-associated protein ubiquitination. CRLs are activated via complex formation with ligase-specific adaptors. Accordingly, PROTACs currently designed with the use of several well-characterized RING ligases, such as Cul2-Rbx-1-EloBC-VHL and Cul4A-Roc1-DDB1-Cereblon, form large complexes involving multiple proteins, including the E3 ligase (Cul2/Cul4A), the adaptor (EloBC/DDB1), and the targeted protein (VHL/Cereblon). A concern is that heavy implementation of PROTACs on these large E3 ligase complexes might lead to a more rapid evolution of the ligase for resistance against the ligands.

By contrast, members NEDD4 subfamily of HECT-type E3 ligases do not require complex formation with other proteins to be active and thus minimize PROTAC complex formation. (EGFR) (Katz et al., Traffic 3: 740-751 (2002); and Weber et al., Front Physiol 10: 370 (2019)) Moreover, PROTACs designed with NEDD4 E3 ligase recruiters can tackle an area of the undruggable proteome left unaddressed by CRL-based PROTACs or other TPD effectors protein systems regulating membrane homeostasis. Assays described herein may be used as a high throughput discovery platform for screening libraries of ligands of membrane proteins for NEDD4 E3 ligase recognition. Through such discovery, NEDD4 E3 ligases may be harnessed to downregulate membrane proteins that are aberrant/upregulated in diseases via TPD.

Moreover, active ubiquitination and degradation of proteins can lead to constitutive activation of signaling pathways, which is the root cause of several classes of diseases, including many tumor suppressors in cancer (for example, TP53, CDKN1A, CDN1C and BAX), and mutated and misfolded proteins, such as AF508-cystic fibrosis transmembrane conductance regulator (CFTR) in cystic fibrosis or glucokinase in pancreatic cells in maturity-onset diabetes of the young type 2. In these cases, a targeted protein stabilization (TPS) therapeutic strategy, rather than TPD, would be appropriate. Here, for example, deubiquitinase (DUB) candidates may be analyzed for their ability to regulate target protein stability.

### EXAMPLES

The following example serve to illustrate the present disclosure. The examples are not intended to limit the scope of the claimed invention in any way.

### Materials

Fmoc-protected amino acids Fmoc-Asn(Trt)-OH, Fmoc-Lys(Boc)-OH, Fmoc-Gln(Trt)-OH Fmoc-Ser(tBu)-OH, Fmoc-Leu-OH, Fmoc-Val-OH, Fmoc-Glu(OtBu)-OH, Fmoc-Pro-OH, Fmoc-Tyr(tBu)-OH, Fmoc-Ala-OH, Fmoc-Arg(Pbf)-OH, and activation agent HBTU (0-benzotriazole-N,N,N′,N′-tetramethyluronium-hexafluoro-phosphate) were purchased from ChemPep, Inc. (Wellington, Florida, USA). ChemMatrix Rink Amide resin was purchased from Gyros Protein Technologies (Uppsala, Sweden). NHS-Fluorescein was purchased from Thermo-Fisher Scientific (Waltham, Massachusetts, USA). N,N-Dimethylformamide (DMF), dichloromethane (DCM), methanol (MeOH), diisopropylethylamine (DIEA), trifluoroacetic acid (TFA), triisopropylsilane (TIPS), and diethyl ether were purchased from Sigma-Aldrich (St. Louis, Missouri, USA). Reagents purchased from commercial sources were used without further purification.

### Peptide Construct Design

The peptide construct was taken from a known PPxY-containing peptide on yeast. Sna4 is a vesicular snare protein with only one lysine which has previously been shown to be ubiquitinated by the membrane protein E3 ligase, Rsp5. The lysine residue is spaced six residues apart from the PPPY recognition motif, hence we decided to synthesize an 11-mer peptide of NKQSLVESPPPY, mimicking residues 128-138 of Sna4, with fluorescein labeled on the N-terminus. The peptide has an expected mass of 1715 Da.

### Solid Phase Peptide Synthesis of Sna4-Peptide

Peptides were synthesized using Fmoc (Fluorenylmethyloxycarbonyl) based solid-phase peptide synthesis on ChemMatrix Rink Amide resin (222 mg, 0.1 mmol), with the reaction automated on the Microwave Peptide Synthesizer Liberty Blue 2.0. The resin was added to the reaction chamber and treated with the desired Fmoc protected amino acid (2 eq., 200 PM), DIC (2 eq., 200 μM) and Oxyma (2 eq., 200 μM) in DMF. This mixture was reacted for 2 minutes at 90° C. The solution was drained from the synthesis chamber and the resin was washed four times with 4 mL DMF. Next, a 20% Piperidine solution in DMF (2 mL) was added to the reaction chamber for 30 seconds at 90° C. to remove the Fmoc protecting group. The piperidine solution was drained and the resin was washed with DMF (2×8 mL each). This process was repeated until all the amino acids in the peptide were coupled on the resin. After the last amino acid deprotection, fluorescein was added to the N-terminus by moving the resin into a peptide synthesis flask and mixed with 5-carboxyfluorescein (2 eq., 0.2 mmol, Fisher Scientific), HCTU (2 eq., 0.2 mmol) and DIEA (6 eq., 0.6 mmol) in DMF for 12 hours.

The peptides were then cleaved from the solid support by adding a solution of 95% TFA, 2.5% TIPS, and 2.5% H2O (10 mL). The filtrate was collected, and the resin was washed with TFA (2×10 mL) and Methanol (2×10 mL). The filtrates were combined, and the solvents were removed under reduced pressure. The peptide was precipitated using cold diethyl ether and collected via centrifugation. The pellet was dried under reduced pressure and the crude mass was determined. The peptide was then resuspended in double deionized water and purified to homogeneity by reverse phase (RP) HPLC on a Luna C18 semi-prep column using a 30-minute linear solvent gradient. Isocratic wash with 5% acetonitrile in water to equilibrate the column for the first 4 minutes, followed by a linear gradient of 5-95% acetonitrile in water for the next 16 minutes. The next 4 minutes is an isocratic wash with 95% acetonitrile in water to completely elute everything from the column. The column is washed with a linear gradient of 95-5% acetonitrile in water for the final 6 minutes.

The masses of the pure peptides were obtained using MALDI-ToF mass spectrometry. The concentrations of the peptides were determined using a UV-Vis spectrophotometer measuring for fluorescein concentration. An absorbance measurement at 495 nm was used to deduce the concentration using the formula (I) (ThermoFisher):

$$[ concentration ] ( M ) = \frac{{A}_{280} - ( {A}_{494} \times 0.3 )}{68000} \times dilution factor \qquad ( I )$$

### Cloning, Expression, and Purification of Rsp5 and NEDD4HYBRID Constructs

Rsp5, NEDD4L-Rsp5HYBRID (No C2 domain), and SdeADUB were cloned into pGEX-6P-1 vector and transformed into E. coli BL21 (DE3) cells to be expressed as GST-tagged proteins. Rsp5 and NEDD4L_{WW2, WW3, WW4}-Rsp5_{HECT} hybrid mutants were generated by site-directed mutagenesis, sequences were confirmed by DNA sequencing and recombinant vectors were transformed into E. coli BL21 (DE3) cells. Ubiquitin cloned into pRSET was transformed into E. coli BL21 (DE3) cells. The full-length construct of NEDD4L was received as a generous gift from Dr. Brenda Schulman (Max Planck Institute). The full-length SMURF2 construct and protein were received as a generous gift from Dr. Ronald Harty (University of Pennsylvania). The full-length WWP2 construct and protein were received as a generous gift from Dr. Philip Cole (Harvard University).

Protein expression was carried out by adding an overnight culture of E. coli cells harboring the appropriate recombinant vector into LB medium supplied with 100 μg/ml ampicillin. Cultures were grown in a shaker incubator at 37° C. until reaching an OD600 of 0.6-0.8. Protein expression was induced by adding 0.35 mM IPTG (isopropyl thio-Dgalactopyranoside) and cultures were then incubated for 16-18 h at 18° C. Cells were harvested by centrifugation at 7,000×g for 6 min at 4° C. and resuspended in (1×PBS pH 7.4, 400 mM KCl) buffer supplied with lysozyme. Resuspended cells were lysed with French press (Thermo Scientific) and then clarified by ultracentrifugation (Beckman Coulter) at 100,000×g for 1 hr at 4° C.

GST-tagged proteins were purified with GST-Sepharose beads (GE Healthcare). Purified proteins were supplied with PreScission™ Protease to cleave the GST tag and dialyzed overnight in (1×PBS pH 7.4, 400 mM KCl, 1 mM DTT) buffer. Dialyzed proteins were then added to GST-Sepharose beads to remove free GST. Proteins were concentrated using Amicon Ultra-15 30K centrifugal columns (Millipore) and protein concentration was determined using NanoDrop A280 (Thermo Scientific).

Ubiquitin in the pRSET-A vector, a gift from the Genentech corporation, was transformed and expressed in a similar manner as Rsp5 and NEDD4L-Rsp5HYBRID. Lysis was performed by subjecting the resuspended cell pellet to in a 90° C. water bath for 30 minutes. Clarified lysate was then produced by ultracentrifugation of the boiled sample (100,000×g for 1 hr). The cleared supernatant was loaded onto a self-packed column of SP Sepharose Fast Flow resin (GE Healthcare) and eluted with a gradient of NaCl, up to 1M. Fractions containing pure ubiquitin, confirmed by SDS-PAGE analysis, were pooled, concentrated, and exchanged into protein storage buffer (50 mM Tris pH 7.4, 50 mM NaCl and 1 mM DTT). Protein concentration was determined using BCA assay.

Sna4 Peptide Ubiquitination in-Gel Fluorescence Assay

0.5 M Rsp5/NEDD4L-Rsp5HYBRID, 100 μm ubiquitin and 10 μM fluorescein-labeled peptides were incubated in the presence or absence of 100 μm ATP for 30 min at 37° C. Reactions were quenched by 5×SDS/PAGE loading buffer and analyzed by SDS-PAGE. In-gel fluorescence was detected using Azure c600 gel imaging system.

### Peptide-Ubiquitination Fluorescence Polarization Assay

Assays were performed by mixing 0.25 m E1 enzyme, 0.5 μm UbcH7, 0.5 μm Rsp5/NEDD4L-Rsp5_{HYBRID}, 100 μM Ub and 10 μM fluorescein-labeled peptide in (50 mM Tris pH 7.4, 100 mM NaCl, 1 mg/ml BSA) buffer. Reaction mixtures were left to equilibrate at room temperature for 10 minutes before taking measurements. Reactions were then started by adding 100 mM ATP. A sample of this reaction was also utilized for SDS-PAGE gel electrophoresis.

Deubiquitination of ubiquitinated Sna4 peptide was monitored by performing a two-step assay. First, the ubiquitination reaction was accomplished using Rsp5/NEDD4L-Rsp5_{HYBRID} and Sna4 peptide as described previously. This was followed by adding SdeA_{DUB} to a final concentration of 10 μm to reverse the ubiquitination reaction 15 minutes into the reaction. A sample of this reaction was also utilized for SDS-PAGE gel electrophoresis.

Inhibition assays were conducted by first incubating 0.5 μm Rsp5/NEDD4L-Rsp5_{HYBRID} with varying concentration of iodoacetamide (12.5-500 μM) for 1 hour in the reaction buffer (50 mM Tris pH 7.4, 100 mM NaCl, 1 mg/ml BSA). Fluorescence polarization was then measured after adding 0.25 μm E1 enzyme, 0.5 μm UbcH7, 100 μm Ub and 10 μm fluorescein-labeled peptide to the reaction well. The Z-factor (Z′) for the assay was calculated using eight replicates of the positive control (no iodoacetamide addition) and negative control (500 μM iodoacetamide addition). The initial slopes of each of the 16 reactions were calculated, followed by calculation of the positive control and negative control means (μ_+ and μ_−) and standard deviations (σ_+ and σ_−). The mean and standard deviation values were then used to calculate the Z-factor using the following formula (II):

$${Z}^{′} = 1 - \frac{3 {σ}_{+} + 3 {σ}_{-}}{\vert {μ}_{+} - {μ}_{-} \vert} \qquad ( II )$$

All assays were carried out at least in triplicate, at a final reaction volume of 100 μL, and utilizing Grenier 96 well plates (Product no. 655097). Fluorescence polarization was measured using a Synergy H4 Plate Reader (BioTek) using 485 nm excitation and 528 nm emission filters.

### Example 1

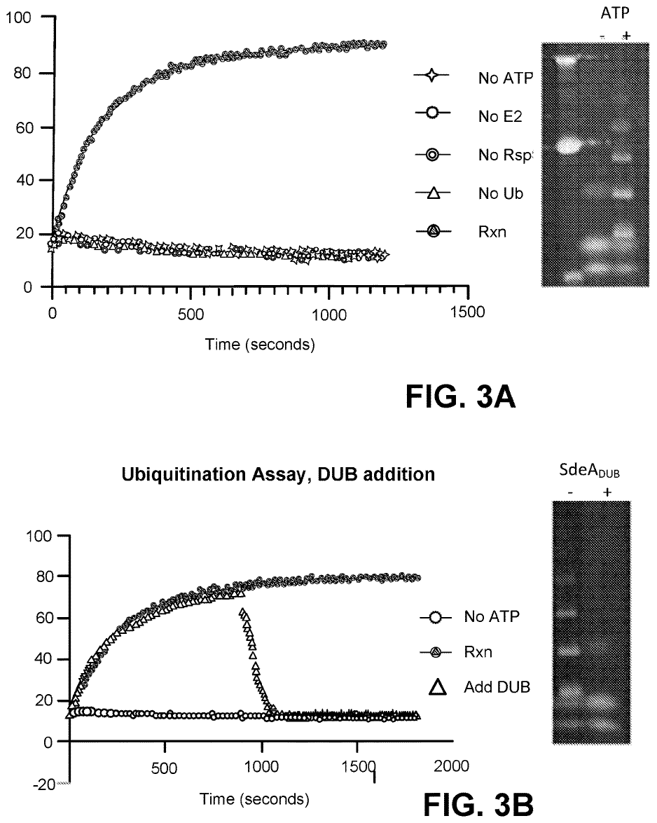

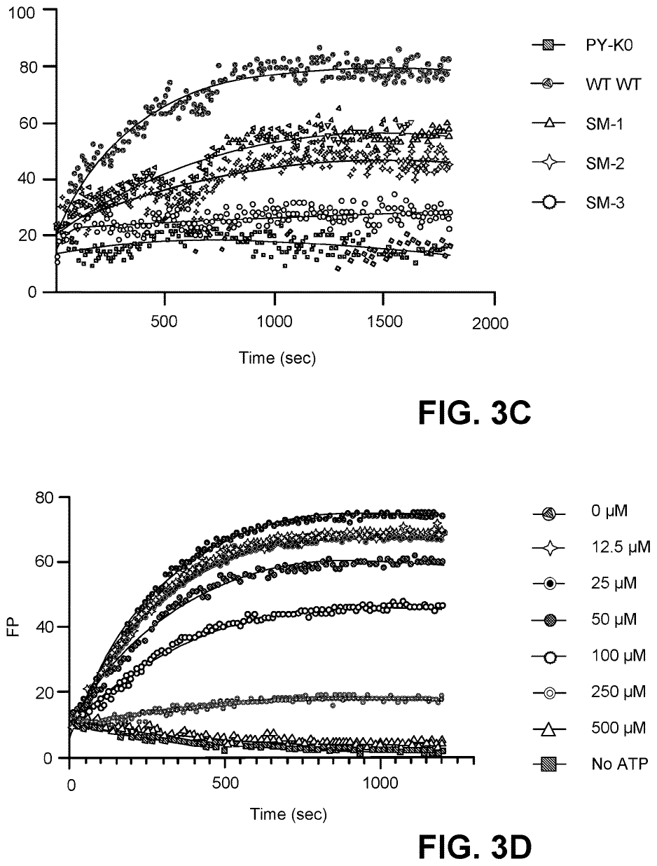

A PPxY peptide based on a yeast vesicular snare protein (Sna4) containing the PPxY motif was designed, synthesized, and labeled with fluorescein at the N-terminus. An assay utilizing Uba1 (E1 ubiquitin (Ub)-activating enzyme), UbcH7 (E2 Ub-conjugating enzyme) and Rsp5 (human E3 Ub-ligase), which lacked the C2 domain to simplify in vitro screening, in the presence of adenosine triphosphate (ATP) and Ub was performed. Fluorescence polarization (FP) was recorded to monitor ubiquitination of the peptide. An increase in fluorescence polarization (λₑₓ=485 nm; λₑₘ=528 nm) was observed in real time (see FIG. 3A). Ubiquitination of the PPxY peptide was confirmed by SDS-PAGE. An in-gel observation of the reaction showed a laddering effect with incremental increases of −8 kDa, indicating the formation of a polyubiquitin chain. No fluorescence polarization occurred in the absence of UbcH7, Rsp5, Ub, or ATP.

Upon the addition of DUB (SdeA_{DUB}), de-ubiquitination occurred, as shown by decreased FP (see FIG. 3B). De-ubiquitination was confirmed by SDS-PAGE.

### Example 2

To confirm that the recognition of the PPxY peptide by Rsp5 was indeed due to the PPxY-motif, analogous peptides were synthesized with the respective proline or tyrosine residue mutated to alanine (peptides APPY, PAPY, and PPPA). It was found that the ubiquitination of the PPxY peptide was indeed specific to Rsp5 (and, by extension, WW-domain containing E3 ligases), as indicated by the significant decrease in FP signal when any of the mutations were made on the PPxY recognition motif (FIG. 3C). This indicates that removal of the second proline and tyrosine are detrimental to the recognition of the PPxY peptide by the WW-domains of Rsp5.

### Example 3

An iodoacetamide inhibition assay to mimic peptide displacement conditions was performed to test robustness and reproducibility. Iodoacetamide irreversibly modifies the catalytic cysteine of the Rsp5 HECT domain, hence decreasing the rate of peptide ubiquitination as observed from the initial slope. This is expected to be the case when more potent competitive inhibitors are added into a reaction. In other words, if a compound is found to interact with the WW-domain with higher affinity than the peptide, the peptide will be displaced, and a lower FP signal will be observed. As such, even among the different compounds in a screening library, different affinities with the WW-domain will be observed with stronger interactors exhibiting lower FP. Adding iodoacetamide at various concentrations mimics this condition, with higher iodoacetamide concentrations mimicking what would be observed in chemical compounds with stronger binding interactions. This was indeed observed when Rsp5 was inhibited with various concentrations of iodoacetamide (FIG. 3D). Furthermore, eight replicates of the no inhibition control (positive control) and the fully inhibited condition (negative control) demonstrated that the results were reproducible with a Z-factor of 0.77.

### Example 4

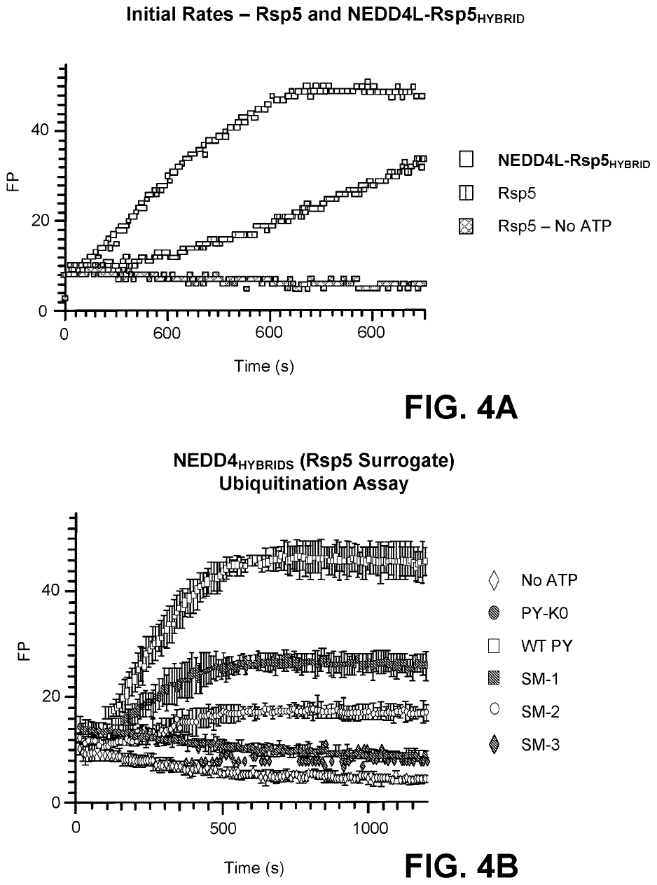

Because assays for binding ligand identification and subsequent usage with PROTACS having human NEDD4 E3 ligase recruiters, the use of NEDD4L under the same conditions applied to Rsp5 was tested. To do so, NEDD4L was recombinantly expressed and purified. Full-length NEDD4L contained one C2 domain, four WW domains, and one HECT domain. Since the ubiquitination of the labeled peptide relies on two stepwise mechanisms as mentioned previously, labeled peptide binding to WW-domain was probed as the measure of ligand binding ability. NEDD4L_{WW2, WW3, WW4}-Rsp5HECT was created in which the WW-domains of Rsp5 were replaced with the WW-domains of NEDD4L. The WW2-WW4 domains of NEDD4L are believed to be more involved in the substrate recruitment, hence NEDD4L_{WW2}, NEDD4L_{WW3}, and NEDD4L_{WW4} was cloned in place of Rsp5_{WW1}, Rsp5_{WW2}, and Rsp5_{WW3}. This hybrid NEDD4L-Rsp5HYBRID construct was observed to have an increased initial rate compared to WT Rsp5 (FIG. 4A). Because the catalytic (HECT) domain is unaltered, this increase in rate is likely due to a tighter binding between the WW domains of NEDD4L to the Sna4 peptide compared to those of the WW domains of Rsp5. The selectivity of the NEDD4L-Rsp5HYBRID construct was then tested to ensure that the protein-peptide interaction is still exclusive to the WW domain-PPxY motif interaction. The peptide ubiquitination reaction was performed using mutants of the peptide and similar results were obtained where a single alanine mutation to any of the key residues (proline or tyrosine) of the PPxY motif significantly decreased the ubiquitination of the peptide (FIG. 4B). Similar results were observed when the assay was performed with other NEDD4 subfamily members, such as SMURF2 and WWP2.

The foregoing assay may be used to screen for deubiquitinases (DUBs) by first ubiquitinating the peptide using the WW-domain HECT type E3 ligase. Candidate DUBs may then be added after the FP signal has reached saturation, around 15 minutes. Upon the addition of DUBs, a decrease in fluorescence polarization (FP) signal will be observed. This decrease in FP is due to the hydrolysis of ubiquitin chain isopeptide linkages which produces the initial peptide before ubiquitination.

The foregoing assay may also be used to screen for DUB inhibitors. To screen for DUB inhibitors, a mixture of DUB and a small molecule inhibitor candidate would first be preincubated. The preincubated mixture may then be added after the FP signal has reached saturation, around 15 minutes. If the small molecule binds and inhibits the DUB, either no decrease in FP signal (best inhibitor) or a slower rate of deubiquitination would be observed, which can be observed by the slope of FP decrease. Under such circumstances, DUB would not be active to perform its isopeptide linkage hydrolysis reaction as effectively, hence keeping the peptides at their ubiquitinated state.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

The invention illustratively described herein may be suitably practiced in the absence of any element(s) or limitation(s), which is/are not specifically disclosed herein. Thus, for example, each instance herein of any of the terms “comprising,” “consisting essentially of,” and “consisting of” may be replaced with either of the other two terms. Likewise, the singular forms “a,” “an,” and “the” include plural references unless the context clearly dictates otherwise. Thus, for example, references to “the method” includes one or more methods and/or steps of the type, which are described herein and/or which will become apparent to those ordinarily skilled in the art upon reading the disclosure.

The terms and expressions, which have been employed, are used as terms of description and not of limitation. In this regard, where certain terms are defined and are otherwise defined, described, or discussed elsewhere in the “Detailed Description,” all such definitions, descriptions, and discussions are intended to be attributed to such terms. There also is no intention in the use of such terms and expressions of excluding any equivalents of the features shown and described or portions thereof. Furthermore, while subheadings, e.g., “Definitions,” are used in the “Detailed Description,” such use is solely for ease of reference and is not intended to limit any disclosure made in one section to that section only; rather, any disclosure made under one subheading is intended to constitute a disclosure under every other subheading.

It is recognized that various modifications are possible within the scope of the claimed invention. Thus, although the present invention has been specifically disclosed in the context of preferred embodiments and optional features, those skilled in the art may resort to modifications and variations of the concepts disclosed herein. Such modifications and variations are considered to be within the scope of the invention as claimed herein.

What is claimed is:

1. A method of identifying a ligand, which is a ligand for a target protein of interest and which interacts with a homologous to E6AP C-terminus (HECT)-type E3 ligase, which method comprises:
(a) incubating the ligand with:
(i) a fluorescently labeled peptide comprising a PPxY motif and
(ii) (a′) an E1 ubiquitin (Ub)-activating enzyme, an E2 Ub-conjugating enzyme, the HECT-type E3 ligase, Ub, and adenosine triphosphate (ATP) or
(b′) a Ub-charged E2 Ub-conjugating enzyme, the HECT-type E3 ligase, and ATP and
(b) measuring fluorescence polarization (FP) over time, whereupon:
if the ligand interacts with the HECT-type E3 ligase, the fluorescently labeled PPxY peptide is displaced from binding to the HECT-type E3 ligase, and there is no change in FP over time and
if the ligand does not interact with the HECT-type E3 ligase, the fluorescently labeled peptide comprising a PPxY motif is ubiquitinated, and there is a change in FP over time,
whereupon a ligand that interacts with a HECT-type E3 ligase is identified.

2. The method of claim 1, wherein the HECT-type E3 ligase is the human NEDD4L.

3. The method of claim 1, wherein the HECT-type E3 ligase is the yeast Rsp5.

4. The method of claim 1, wherein the HECT-type E3 ligase is a hybrid in which the WW1-WW3 domains of Rsp5 have been replaced with the WW2-WW4 domains of NEDD4L.

5. The method of any one of claims 1-3, wherein the fluorescently labeled peptide comprising a PPxY motif is a fluorescently labeled peptide having an amino acid sequence from yeast vesicular snare protein (Sna4) and containing a PPxY motif.

6. A composition comprising (a) a fluorescently labeled peptide comprising a PPxY motif, an E1 ubiquitin (Ub)-activating enzyme, an E2 Ub-conjugating enzyme, a HECT-type E3 ligase, Ub, adenosine triphosphate (ATP), and a reaction buffer or (b) a fluorescently labeled peptide comprising a PPxY motif, a Ub-charged E2 Ub-conjugating enzyme, a HECT-type E3 ligase, ATP, and a reaction buffer.

7. A composition comprising the ligand identified in accordance with the method of any one of claims 1-5 or a pharmaceutically acceptable salt, enantiomer, stereoisomer, solvate, or polymorph thereof.
